(19) United States
(12) Patent Application Publication
(10) Pub. No.: US 2008/0038733 A1
(43) Pub. Date: Feb. 14, 2008

(54) SCREENING FOR DOWN SYNDROME

(75) Inventors: Farideh Z. Bischoff, Sugarland, TX (US); Joe Leigh Simpson, Miami, FL (US)

Correspondence Address: FULBRIGHT & JAWORSKI, LLP, 1301 MCKINNEY, SUITE 5100, HOUSTON, TX, 77010-3095, US

(73) Assignee: BAYLOR COLLEGE OF MEDICINE, Houston, TX (US)

(21) Appl. No.: 11/692,115

(22) Filed: Mar. 27, 2007

Related U.S. Application Data
(60) Provisional application No. 60/786,660, filed on Mar. 28, 2006.

(51) Int. Cl. C12Q 1/68

(52) U.S. Cl. 435/6

(57) ABSTRACT

The present disclosure describes methods for screening and identifying genomic sequences useful in estimating the risk of fetal aneuploidy, particularly trisomy 21. This disclosure also describes methods for utilizing such genomic sequences alone or to augment existing non-invasive diagnostics for Trisomy 21 and other aneuploidies.

### CROSS-REFERENCE TO RELATED APPLICATIONS

This Application is a Regular Utility Application claiming the benefit of Provisional application 60/786,660 filed on Mar. 28, 2006, pursuant to 35 U.S.C. 119(e). This provisional application is hereby incorporated by reference in its entirety.

### STATEMENT REGARDING FEDERALLY SPONSORED RESEARCH OR DEVELOPMENT

The work herein was supported by NIH/NICHD grant HDO46623. The United States Government may have certain rights in the invention.

### TECHNICAL FIELD

The filed of invention is diagnostic testing for genetic disorders, specifically estimations of the risk of fetal aneuploidy based on indirect parameters.

### BACKGROUND OF THE INVENTION

Chromosomal abnormalities occur in 0.1% to 0.2% of live births. Among these, the most common, clinically significant abnormality is Down syndrome (Trisomy 21). Currently there are both diagnostic and screening tests for chromosomal abnormalities, but unfortunately, all of them have serious limitations. The diagnostic tests involve small but significant risks to the fetus and mother in obtaining the needed fetal cells, and the screening tests suffer from less than desirable sensitivity and/or specificity. Because of these limitations, a great deal of effort is currently being directed toward the development of improved screening and diagnostic tests. One approach that is being explored in several laboratories is to isolate fetal cells from the mother's blood, and to use the DNA from these cells for prenatal diagnosis. Such a test would have compelling advantages over those that are currently available as it would be noninvasive and could be done very early in pregnancy. The major problem that must be overcome to render this approach feasible is the effective isolation of the fetal cells, which are present in the mother's blood in very small numbers.

Cytogenetic analysis is one diagnostic test available for chromosomal abnormalities, including Trisomy 13, Trisomy 18, Klinefelter syndrome, XYY, Turner syndrome and Down syndrome. This highly accurate and well-established test has a major disadvantage in that it requires an invasive procedure, either amniocentesis or chorionic villus sampling (CVS) to obtain fetal tissue. This presents three problems: (1) risk to both the fetus and the mother, (2) delay in diagnosis, and (3) cost. Because amniocentesis and CVS are invasive procedures, there is a small but significant risk to the fetus and a slight risk of infection for the mother. Amniocentesis is generally done at 15 weeks of gestation, although at some centers it is performed as early as 11-14 weeks. Chorionic villus sampling is done at 9-12 weeks gestation. The earlier diagnosis afforded by CVS or early amniocentesis is advantageous because of reduced emotional stress on the parents and medical advantages associated with an early termination of pregnancy should the parents so choose. However, earlier diagnosis entails an increased risk to the fetus.

The risk of fetal loss is small but significant. It is generally quoted that there is about a 0.5% risk of fetal loss as a consequence of a second-trimester (16 week) amniocentesis. The risk associated with early amniocentesis (14 weeks) or CVS is somewhat greater. For women under age 35 without a predisposing factor, the risk of fetal loss due to amniocentesis is felt to be greater than the incidence of Down syndrome; thus, a diagnostic test is often only recommended for women 35 or over unless there is another predisposing factor. The most common predisposing factor is a positive screening test. For women over 35, the incidence of Down syndrome increases rapidly with increasing age. At age 35, the incidence may be about one in 200 live births; it increases to about one in 46 at age 45. Although the risk of Down syndrome (as well as other chromosome abnormalities) is greatly increased, the consequences of a fetal loss due to amniocentesis are also much greater, since these older women may not be able to achieve another pregnancy.

Because of the risks associated with the prenatal diagnostic tests currently available, a large amount of effort has been dedicated towards developing more effective screening tests. Whereas the diagnostic test is a highly accurate and sensitive way of detecting chromosomal aneuploidies, the screening tests that are currently available provide only an indication of whether or not a fetus is affected with Down syndrome or another chromosomal abnormality. Multiple non-invasive options exist in both first and second trimester (Malone et al., 2005; Simpson, 2005). These non-invasive screening methods can identify 80-93% of trisomy 21 fetuses, given a 5% procedure (false positive) rate. A negative result from a screening test does not necessarily mean that the child will be unaffected (only that there is a lower risk), and a positive result must be followed up by the diagnostic test to be meaningful. Because of the relatively low specificity of the current screening tests and the requirement that positive tests be validated by a diagnostic cytogenetic test, a large number of normal pregnancies continue to be jeopardized by amniocentesis. To increase sensitivity, or lower procedure rate, additional non-invasive screening analytes are continually sought.

There are two types of screening tests generally now available: a blood test conducted on the mother, and an ultrasound test conducted on the fetus. The blood test is generally done in the second trimester, typically between 15 and 20 weeks gestation. In this test, a blood sample is taken from the mother and the levels of one, two, three or four biochemical markers are determined. This test is referred to as a “triple screen” if three markers are determined, or a “quad screen” if four markers are determined. The results of these tests also serve as a screening test for Trisomy 18 and for neural tube defects.

The use of a triple screen for pregnant women under age 35 may be the current standard of practice covered by many insurance companies. The markers that are measured in the triple screen are alpha-fetoprotein, chorionic gonadotropin, and unconjugated estriol. Recently, a fourth biochemical marker, inhibin-A, has been added to the triple screen to form the “quad screen.”

The triple screen has been in use for a number of years, and a considerable amount of data on the sensitivity and specificity of the test has been accumulated. Sensitivity and specificity vary with the age of the mother and with the cutoff criteria used by the various investigators. Generally, out of 1000 women tested, about 100 will test positive, i.e. meaning a recommendation will result to follow up with amniocentesis for a cytogenetic study. Of this 100, only two or three will actually have a fetus with Down syndrome. Of the 900 who test negative, about two will have a child with Down syndrome. Thus, many providers do not believe that this test truly provides a woman with greatly increased assurance of a child without Down syndrome; instead it is felt that it subjects many couples to the emotional stress associated with receiving a positive test and also subjects many normal fetuses to the risks of amniocentesis.

Second-trimester ultrasound screening has alternatively become a routine part of prenatal care in many practices, and several sonographic markers have been associated with chromosomal abnormalities. However, review of studies conducted for over a decade found that, in the absence of associated fetal abnormalities, the sensitivity of these markers was low and that there was a relatively high false positive rate in detecting Down syndrome.

It has been realized for some time now that fetal cells are present in the mother's blood, and that these cells present a potential source of fetal chromosomes for prenatal DNA-based diagnostics. (Price et al., 1991; Elias et al., 1992; de la et al., 1995) Because these cells appear very early in the pregnancy, they could form the basis of an accurate noninvasive first trimester test. A number of methods for isolating these cells have been proposed, however, enrichment methods remain complex and inefficient in the absence of a fetal specific marker. As a consequence, clinical feasibility has not yet been demonstrated. Experimental attempts using fetal cells from maternal blood to identify fetal chromosomal aneuploidy (trisomy 21) in first and second trimester pregnancies at best detects 75% of cases with a false-positive rate estimated between 0.6 and 4.1% (de la et al., 1998; Bianchi et al., 2002).

U.S. Pat. No. 5,252,489 discloses a screening test for Down syndrome and perhaps other chromosomal anomalies to determine whether a pregnant woman's risk of carrying a fetus with Down syndrome warrants further testing. The test procedure can utilize a few drops of blood from a prick at the tip of a finger, an earlobe or the like, which are collected on a piece of filter paper or the like. The test relies upon comparison of the level of free Beta HCG in the dried blood spot against reference values of the level of free Beta HCG accumulated by testing women during similar gestational periods who then experienced either normal childbirth or a child or fetus diagnosed with a chromosomal anomaly such as Down syndrome. Based upon this comparison, a risk assessment is made to allow the pregnant woman to decide whether she should then undergo diagnostic testing or whether the risk appears to be so low that further testing is unwarranted. Although the concept of such screening is good, it may not be more effective than the previously described triple screen and quad screen, and it has not achieved wide acceptance because the results have not been shown to be sufficiently accurate to provide parents with a greatly increased assurance of whether the fetus is or is not affected with Down syndrome.

Accordingly, the search has gone on for simple, straightforward and more accurate screening tests that can be non-invasively performed on a pregnant woman, preferably in the first trimester.

Cell-free fetal DNA (cffDNA) exists in plasma of pregnant women as early as six weeks of gestation. Concentrations rise during pregnancy and peak prior to parturition (Lo et al., 1998). Many laboratories, including ours, have shown the utility of fetal circulating DNA as a unique source of genetic material for non-invasive prenatal evaluation of fetal gender, genetic diseases, and aneuploidy using quantitative PCR (Bischoff et al., 1999; Bischoff et al., 2002; Bischoff et al., 2003; Bischoff et al., 2005; Johnson et al., 2004). Moreover, cell free DNA has been used to distinguish euploid from trisomy 21 pregnancies. Studies indicate increased levels of cell-free fetal DNA in trisomy 21. These studies have been limited to pregnancies carrying a male fetus, because only Y chromosome sequences can be reliably distinguished from maternal DNA. (Lo et al., 1999; Zhong et al., 2000; Ohashi et al., 2001; Lee et al., 2002; Hromadnikova et al., 2002; Spencer et al., 2003; Farina et al., 2003; Bauer et al., 2006). These Y chromosome specific results do not address the need for a gender independent marker based on cff-DNA.

### BRIEF SUMMARY OF THE INVENTION

The methods disclosed herein may be described briefly by the following:

A method of identifying genomic DNA sequences that are useful for estimating the probability of an aneuploid pregnancy, the method comprising the steps of estimating a total concentration of a non-Y chromosome DNA sequence in a bodily fluid sample representing an aneuploid fetal pregnancy, estimating a total concentration of a non-Y chromosome DNA sequence in a bodily fluid sample representing a euploid fetal pregnancy, and comparing the total concentration estimates for the DNA sequence(s) to determine whether the estimated concentration representing a euploid fetal pregnancy are significantly different from the estimated concentration representing an aneuploid fetal pregnancy.

The method of paragraph [0017], wherein the bodily fluid is whole blood.

The methods of paragraphs [0017] and [0018] further comprising the step of purifying the genomic DNA from the sample.

The methods of paragraphs [0017] to [0019], wherein the total concentration of a non-Y chromosome DNA sequence is estimated using real time polymerase chain reaction.

The methods of paragraphs [0017] to [0020], wherein the comparison in [0017] comprises one or more of comparing the mean values and their standard deviations, or comparing the median values of the total concentration estimates for the DNA sequence(s) to derive a Multiplicity of the Median value for the total concentration of a non-Y chromosome DNA sequence in a bodily fluid sample representing an aneuploid fetal pregnancy.

The method of paragraph [0021], wherein the total concentration estimates are first log converted and the conversion to a Multiplicity of the Median value is performed by using a weighted log-linear regression.

The methods of paragraphs [0017] to [0022], further comprising the steps of comparing the total concentration estimates for the DNA sequence(s) to a predetermined first trimester risk estimate for aneuploidy, and determining if the total DNA concentration estimates are significantly correlated with the predetermined first trimester risk estimate.

A method of estimating the probability of a test bodily fluid sample representing an aneuploid fetal pregnancy, the method comprising the steps of estimating a total concentration of a non-Y chromosome DNA sequence in the test bodily fluid sample, comparing the estimated concentration with a control data set representing an expected total concentration of a non-Y chromosome DNA sequence in bodily fluid samples representing euploid fetal pregnancy, and determining, based on the comparison, a probability that the test bodily fluid sample represents an aneuploid fetal pregnancy.

The method of paragraph [0024], wherein the control data set is matched to the test bodily fluid sample for one or more of gestational age, maternal age, maternal weight, maternal diabetic status, maternal smoking status, prior maternal history of aneuploid pregnancy and maternal race.

The methods of paragraphs [0024] and [0025] wherein the comparison referred to in paragraph [0024] is performed by calculating the Multiplicity of the Median for the total concentration of a non-Y chromosome DNA sequence in the test bodily fluid sample, and the determination referred to in paragraph [0024] is based on whether the Multiplicity of the Median value meets or exceeds a threshold value which has been empirically determined to correspond to a probability of aneuploidy.

The method of paragraph [0026] wherein the Multiplicity of the Median value corresponding to a probability of aneuploidy is at least about 1.3 if calculated directly, or at least about 1.5 if calculated using log converted data and a weighted log-linear regression.

The methods of paragraphs [0024] to [0027], wherein the non-Y chromosome DNA sequence in the test bodily fluid sample comprises a Beta-globin genomic locus sequence.

The method of paragraphs [0028], wherein the Beta-globin genomic locus sequence is amplified by polymerase chain reaction primers comprising SEQ ID NO: 7 and SEQ ID NO: 8.

The methods of paragraphs [0024] to [0029], wherein the test bodily fluid sample corresponds to a first trimester pregnancy.

A method of estimating the probability of a bodily fluid sample representing an aneuploid fetal pregnancy, the method comprising the steps of estimating a total concentration of a non-Y chromosome DNA sequence in the bodily fluid sample, comparing the estimated concentration with a control data set representing an expected total concentration of a non-Y chromosome DNA sequence in bodily fluid samples representing euploid fetal pregnancy, determining, based on the comparison, a probability that the bodily fluid sample represents an aneuploid fetal pregnancy, and combining the determined probability with additional probability estimates of aneuploid fetal pregnancy to derive a combined probability estimate.

### BRIEF DESCRIPTION OF THE DRAWINGS

FIG. 1 shows DNA concentrations in maternal whole blood DNA for: Beta-globin, GAPDH, Beta-actin and p53. Trisomy-21 (tri21) and euploid (eup) groups are indicated on x-axis. DNA concentrations are expressed in genome-equivalent/ml and are plotted on the y-axis. The mean in each group is indicated by a line;

FIG. 2 shows Beta-globin levels in trisomy-21 and matched age euploid pregnancies. Values from maternal whole blood DNA of trisomy-21 are represented by squares and age matched euploid group by triangles. Gestational age (weeks) is indicated on x-axis. Concentrations of DNA are expressed in genome-equivalent/ml and are plotted on the y-axis.; and

FIG. 3 shows curves representing the distribution of individual MoM values using the Beta-globin locus measurements from Table 1.

### DETAILED DESCRIPTION OF THE INVENTION

Definitions

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having”, “including” and “comprising” are interchangeable and one of skill in the art is cognizant that these terms are open ended terms.

The present disclosure describes methods for screening and identifying genomic sequences useful in estimating the risk of aneuploidy, particularly trisomy 21. This disclosure also describes methods for utilizing such genomic sequences alone or to augment existing non-invasive diagnostics for Trisomy 21 and other aneuploidies. Common steps in both types of methods may include maternal blood collection, DNA purification and determining the concentration of DNA in maternal blood, as described below.

Blood Collection

The techniques described herein generally involve the analysis of DNA from maternal blood. The preferred procedures described herein involve the analysis of DNA from dried blood spots. Blood may be collected from pregnant women and dried blood spots may be produced in a variety of ways as will be apparent to one of skill in the art. The most preferred methodology is to collect blood from a finger prick and spot blood onto standard blood specimen cards. Blood specimens may be further treated in a variety of ways such as preserving by paraformaldehyde treatment, freeze drying a filter/dried blood spot, or air drying and vacuum sealing the blood specimen. The following working example is one embodiment of this most preferred method.

### EXAMPLE

### Blood Collection

Drops of blood from a finger stick were spotted onto a sterile S&S 903 specimen collection card (Schleicher and Schuell, Keene, N H, lot #W0131). Filter cards containing blood samples were all coded and allowed to air dry overnight then transferred to sterile specimen plastic bags for storage at room temperature.

DNA Purification

Several commercially available kits allow isolation of DNA from bodily fluids and DNA may be purified from maternal blood by a variety of methods. Techniques for extraction of DNA from whole blood, blood serum, or blood plasma are well known in the art. Swinkels et al., 2002; Spencer et al., 2003; Bischoff et al., 2003. The preferred DNA purification starting material is whole blood. Most preferred, the whole maternal blood is in the form of a dried blood spot. The following working example is one embodiment of this most preferred method.

### EXAMPLE

### DNA Purification

Six 3 mm punched-out circles were taken from blood spots on S&S 903 specimen collection cards (Schleicher and Schuell, Keene, N H, lot #W0131). These punch outs were placed together in a 1.5 ml microcentrifuge tube. Based on product information, 100 ul of blood is estimated to be absorbed per 16 mm diameter of filter paper. Therefore, for the six 3 mm punched out sections, 112 ul of blood was estimated to be used for DNA extraction. DNA was extracted using the QIAAMP® DNA blood kit for dried blood spots (QIAGEN®, Valencia, Calif. USA) according to the manufacturer's instructions. All DNA samples were stored at 4° C. until analysis.

DNA Quantitative Analysis

Purified DNA from maternal blood may be measured by a variety of methods known in the art. In circumstances where sufficient DNA is present, direct spectroscopic measurement or indirect fluorescence measurements of intercalating dyes may, for example, be used. However, the preferred methods generally use small amounts of DNA not suited to direct measurements. In such cases, DNA may be quantified by a number of alternative techniques. Any method that enables measurement of fluorescent signal as it is generated as result of amplification may be used with the methods herein. The most preferred technique for measuring DNA quantities is real-time polymerase chain reaction (RTPCR). In this most preferred method, PCR primers capable of amplifying a genomic DNA sequence are used to amplify purified DNA from maternal blood and, in parallel, a control DNA having the same target sequence present in a know amount. These amplification reactions generally occur in the presence of an intercalating dye, most often SYBR green, which allows continuous fluorescent detection of the amplification products. Alternatively, the amplification primers may be supplemented with a target sequence probe that incorporates fluorescent moieties released during amplification (“TAQMAN® Assay”). These released fluorescent moieties allow more specific detection of amplification products. Comparison of the measurements of a titration series of control DNA to the purified maternal DNA measurements allows for determination of the amount of original target genomic DNA in the purified DNA sample. The general principles of RTPCR are well know to those of skill in the art as is discussed in detail in Real-Time PCR: An Essential Guide, Editor: Kirstin Edwards, Julie Logan and Nick Saunders, Horizon Bioscience (2004) ISBN-13: 978-0-9545232-7-5, the contents of which are incorporated herein by reference.

### EXAMPLE

### DNA Quantitative Analysis

Quantitative real-time PCR using a TaqMan Assay to measure total DNA levels of four non-chromosome 21 loci: glyceraldehyde-3-phosphate dehydrogenase GAPDH (12p13) (GenBank Accession No. NC_{—}000012); Beta-globin (11q21) (GenBank Accession No. NC_{—}000011); Beta-actin (7ptel); and p53 (17p13) (GenBank Accession No. NC_{—}000017) were performed using the APPLIED BIOSYSTEMS® 7700 sequence detection system.

PCR primers and probes were as follows:

GAPDH forward

(SEQ ID NO: 1)

5′-CCC CAC ACA CAT GCA CTT ACC-3′
GAPDH reverse

(SEQ ID NO: 2)

5′-CCT AGT CCC AGG GCT TTG ATT-3′
GAPDH fl probe

(SEQ ID NO: 3)

5′-6FAM-AAA GAG CTA GGA AGG ACA GGC AAC TTG GC-
TAMRA-3′
P53 forward

(SEQ ID NO: 4)

5′-GGT CGG CGA GAA CCT GACT-3′
P53 reverse

(SEQ ID NO: 5)

5′-CTG CCG GAG GAA GCA AAG-3′
P53 fl probe

(SEQ ID NO: 6)

5′-6FAM-TGC ACC CTC CTC CCC AAC TCCA-TAMRA-3′
Beta-Globin forward

(SEQ ID NO: 7)

5′-GTG CAC CTG ACT CCT GAG GAGA-3′
Beta-Globin reverse

(SEQ ID NO: 8)

5′-CCT TGA TAC CAA CCT GCC CAG-3′
Beta-Globin fl probe

(SEQ ID NO: 9)

5′-6FAM-AAG GTG AAC GTG GAT GAA GTT GGT GG-TAMRA-
3′

Beta-actin primer probes (proprietary sequences) were purchased from APPLIED BIOSYSTEMS® (TAQMAN® Beta-actin Control Reagents, APPLIED BIOSYSTEMS® Part Number: 401846).

For each amplification reaction (50 μL), we used 300 nM each primer, 160 nM probe, 25 μL of TAQMAN® Universal Master Mix (APPLIED BIOSYSTEMS®), and 5 μL of DNA sample. After 10 min at 95° C., 40 two-step cycles were performed (30 s at 95° C. and 1 min at 60° C.).

Cycle threshold values for each locus tested were presented by the computer and were transformed to copy numbers with calibration curves of known concentrations of human genomic DNA (Sequence Detection Systems 1.7.1; 6.6 pg of DNA=2 copies=1 cell-equivalent). Quantification of DNA as genome equivalents per ml of blood was based on copies of the gene sequences detected per microliter of blood. Each reaction plate was run simultaneously with a duplicate calibration curve of titrated DNA (standard curve). Each sample (5 μl) was run in triplicate for each locus, and the mean of the values was determined using the 7700 software and the standard curve of known DNA concentrations. All samples were analyzed blindly with respect to genotype (trisomy 21). Control specimens included filter paper alone (processed without blood), and PCR blanks (no DNA).

Screening and Identifying Genomic Sequences Useful in Estimating the Risk of Aneuploidy.

cffDNA as well as fetal cellular DNA are present in maternal blood. A consequence of this phenomenon is that shared genomic sequences show elevated total concentrations in maternal blood. It has been shown using Y chromosome specific genomic sequences that fetal derived DNA is further increased in maternal blood when the fetal karyotype is trisomy 21. In principle, this relative increase in fetal DNA should also be discernable as a relative increase in total maternal blood DNA. However, statistically significant and reproducible measurements of the elevation of total genomic DNA in women with euploid versus aneuploid pregnancies have proved elusive. Thus, the following screening method is designed to identify genomic sequences which are, on average, elevated in maternal blood in aneuploid pregnancies. Genomic sequences with potential for use as diagnostic factors in indirect risk assessments for aneuploidy are those sequences elevated in total concentration in maternal blood to a statistically significant extent over euploid pregnancies.

The screening process generally proceeds as follows: A genomic sequence is selected; primers and probes are designed to amplify and detect the genomic sequence using Real-time PCR; optionally, Real-time PCR conditions are optimized for each specific primer pair; purified total maternal blood DNA from both euploid and aneuploid cases are amplified to measure the concentration of the specific target genomic DNA sequence. The relative concentration of the specific target genomic DNA sequence is subjected to statistical analysis to determine if the difference in concentration between euploid and aneuploid is statistically significant.

Primer Design

Primer sequence selection and primer/probe set designs may be performed using a variety of techniques well known in the art. Generally, the primer melting temperature (Tm) of each PCR primer should be between 58-60° C., and any TAQMAN® probe Tm should be ˜10° C. higher than the primer Tm. The Tm of both primers should generally be within 1° C. The primers for the working examples were designed using APPLIED BIOSYSTEMS® PCR PRIMER EXPRESS® Software used to create best primer/probe sets targeting the selected genomic loci. However, many other computer assisted primer set design methods are available to one of skill in the art.

RTPCR Optimization

One may empirically determine optimal reaction parameters for each assay, such as primer concentrations, enzyme amounts, Mg²⁺ concentrations, annealing temperatures and times through routine experimentation. The working examples provided generally conform to the standard conditions recommended by the manufacturer of the RTPCR “universal master mix” (APPLIED BIOSYSTEMS®) used.

Purified Maternal Blood DNA

Maternal blood and total maternal blood DNA are purified as described above. The preferred methods of blood collection and DNA purification described previously are also the preferred methods for use in screening genomic sequences.

DNA Quantitative Analysis

Total maternal blood DNA is analyzed for the quantity of specific genomic sequences as described above. The TAQMAN® RTPCR method described previously is also the preferred method for use in screening genomic sequences.

Statistical Verification

The total concentrations of maternal blood DNA in euploid and aneuploid cases are compared to determine if the increased concentration in aneuploid cases is statistically significant. The preferred initial statistical evaluation is a two tailed t test to compare euploid and aneuploid DNA levels. Data should generally be stratified to match maternal age, gestational stage and maternal weight. Exemplary data produced using the preferred TAQMAN® RTPCR method are shown in Table 1.

TABLE 1
Comparison of different parameters between women
carrying euploid fetus vs. trisomy 21 fetus.

| | Eulpoid | | Trisomy 21 | |
|---|---|---|---|---|
| n | 30 | | 17 | |
| Maternal age | 32.33 ± 5.02 | (16-43) | 37.18 ± 3.86 | (28-43) |
| (years) | | | | |
| Gestational age | 12.30 ± 0.78 | (11.0-13.8) | 12.23 ± 0.77 | (11.0-13.8) |
| (weeks) | | | | |
| Maternal weight | 140.80 ± 22.46 | (111-198) | 144.24 ± 20.86 | (109-175) |
| (pounds) | | | | |
| First trimester risk | 3448.00 ± 2714.50 | (158-10000) | 270.82 ± 527.88 | (5-1830) |
| Second trimester risk | 2798.41 ± 2612.52 | (89-10000) | 38.50 ± 46.98 | (6-130) |
| | n = 23 | | n = 6 | |
| GAPDH | 1.76 ± 1.06 | (0.16-4.68) | 2.24 ± 1.03 | (0.91-3.59) |
| (Geq/ml × 10⁵) ^{a} | | | | |
| Beta-actin | 0.79 ± 0.78 | (0.01-2.61) | 1.01 ± 0.64 | (0.24-2.42) |
| (Geq/ml × 10⁵) ^{b} | | | | |
| Beta-globin | 2.35 ± 1.84 | (0.10-7.28) | 4.08 ± 1.78 | (1.76-6.51) |
| (Geq/ml × 10⁵) ^{c} | | | | |
| p53 | 1.59 ± 1.09 | (0.26-4.26) | 2.27 ± 1.15 | (0.73-4.30) |
| (Geq/ml × 10⁵) ^{d} | | | | |

Values are expressed as mean±SD with the range of values in parenthesis; Euploid vs. Trisomy 21: ^{a}p=0.179; ^{b}p=0.346; ^{c}p=0.003; ^{d}p=0.05.

SPSS® 11.0 software was used for this and all other statistical calculations. There were no significant differences in age, weight or gestational age among the trisomy 21 and control groups (p<0.05) in this example. Normality of the Beta-globin locus measurements was tested by Kolmogorov-Smirnov Z test. p values were 0.772 and 0.256 for controls and cases, respectively, and thus the distributions are normal. Variances of the Beta-globin locus measurements were 0.22 and 0.04 for the control and trisomy 21 data, respectively, in Table 1.

As expected, a significant difference between the levels of first trimester risk based on standard quad factor serum screening and Neural Tube (NT) measurements for the two groups (p=0.00003) was observed. As demonstrated by the subsequent analysis below, potentially useful genomic sequences generally show a significant difference with p≦0.025, more preferably p≦0.01. Thus, for example, the p53 sequence with a p level of 0.05 would not be expected to prove useful as a diagnostic factor for trisomy 21. However, p=0.05 is the generally accepted minimum p value indicating statistical significance. Thus, it is contemplated that a marker with at least p=0.05 would be further analyzed as described below.

While, the initial screening evaluation step is generally sufficient, additional statistical analysis is preferred. One optional statistical evaluation is to evaluate the relationship of elevated genomic DNA to gestational age. Preferably, relatively elevated DNA should be seen within a clinically useful gestational age range, preferably within at least a three week window, more preferably from week 11 to week 14. The elevated DNA most preferably results in at least 30%, more preferably about 50% or more, of the euploid DNA concentration values falling below matched trisomy 21 DNA concentration values. It is also preferred that less than 50%, more preferably less than 25% of the euploid DNA concentration values fall above the mean of all trisomy 21 DNA concentration values in a data set (e.g. the mean in Table 1).

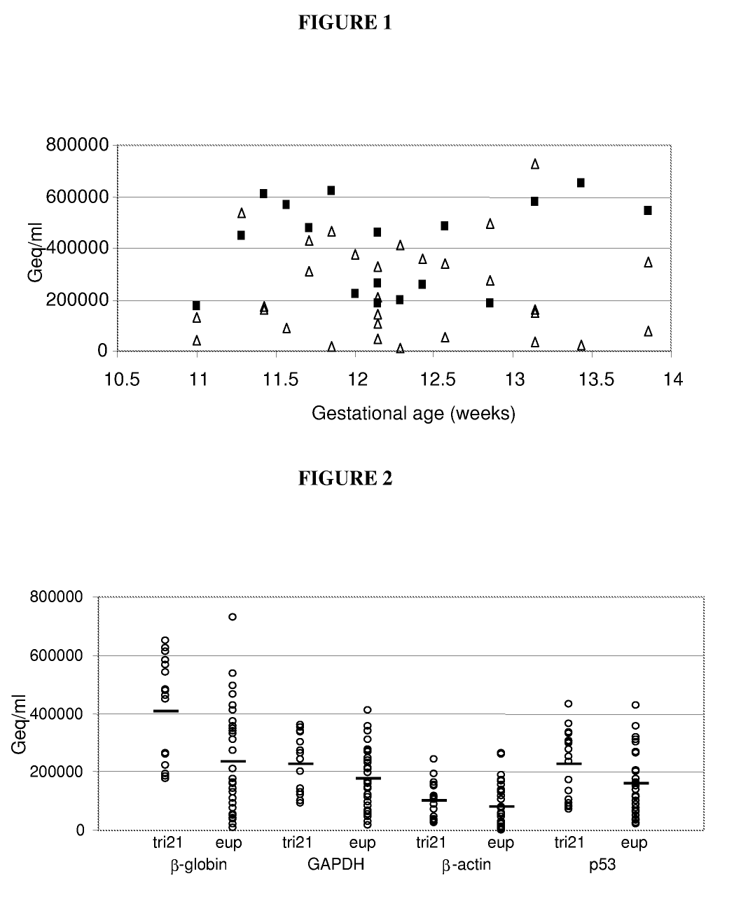

FIG. 1 shows the exemplary data from Table 1, charted against gestational age. Results from maternal-dried blood spots (“DBS”) of trisomy-21 are represented by squares and age matched euploid group by triangles. Gestational age (weeks) is indicated on x-axis. Concentrations of DNA are expressed in genome-equivalent/ml and are plotted on the y-axis. All 17 confirmed trisomy 21 cases were matched blindly, by gestational age to 1 or 2 euploid control cases. In 13 of the trisomy 21 cases, a significant difference was observed between the levels of Beta-globin versus the matched euploid control (p-values in the full data set ranging from 0.9401 to 0.0003). Though overlap exists between the values observed in trisomy 21 and euploid pregnancies (FIGS. 1 and 2), 50% of the euploid samples had Beta-globin values lower than the matched trisomy 21 samples, whereas only 20% of the euploid samples had Beta-globin values higher than the mean values for trisomy 21 samples (FIG. 2).

An additional preferred statistical verification analysis is conversion to and analysis based upon Multiplicities of the Median (MoM). MoM is a statistical convention introduced into clinical testing as a method for laboratories to compare individual test results. Measurements of many clinical values can be affected by specific laboratory techniques, resulting in difficulty comparing absolute results between laboratories. Reliance on standard deviations is often suboptimal because standard deviation calculations are influenced by data spread. Because MoM is calculated from of an individual sample value compared to the median of a control data set, it is not influenced by outlying values in the control group (data spread) in the way standard deviation calculations are. Each laboratory should develop reference data, with a median value from unaffected pregnancies calculated for each week of gestation. However simplified control data values, consolidated over multiple gestational ages, may be utilized if the control data is shown to be consistent across a range of such ages (e.g. weeks 11-14). Preferably, the control data is stratified according to various other variables known or suspected of influencing the median value for total maternal blood DNA. These other variables may include maternal age, weight, race, multiple gestations and insulin dependent diabetes mellitus.

Total maternal blood DNA is expressed as a MoM by dividing the total maternal blood DNA concentration measured in a specific sample by the median control data value, generally for the appropriate week of gestation. The median value for the control data for total maternal blood DNA is by definition 1.0 MoM. The mean trisomy 21 value for the Beta-globin measurements converts to a MoM of approximately 1.7 whereas measurements using the p53 locus gives a MoM of approximately 1.4. Preferably, a genomic DNA sequence useful for screening for aneuploidies yields a MoM of at least about 1.3, more preferably at least about 1.5 and most preferably at least about 1.7.

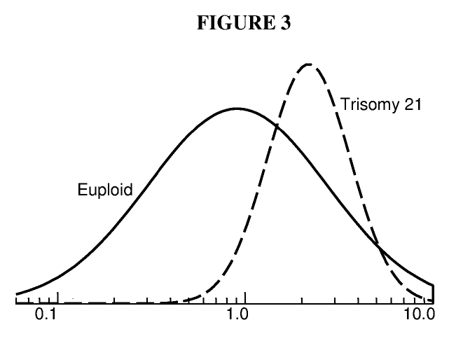

Alternative MoM derivations are both feasible and contemplated for use with the methods herein. One such alternative MoM calculation is 1) log conversion of total maternal DNA measurements and 2) conversion to MoM values by using a weighted log-linear regression. Data derived by this alternative calculation is represented by the lines in FIG. 3 using the Beta-globin locus measurements from Table 1. The MoM calculated in trisomy 21 cases using this alternative calculation was 2.8 for the exemplary Beta-globin locus, compared to 1.8 for the p53 locus (and 1.0 by definition for the control samples). The distribution of circulating DNA levels in both trisomic and euploid pregnancies followed a log-Gaussian pattern at least between the 10th and 90th centiles of unaffected, as judged by a probability plot. Normality for the exemplary Beta-globin locus results was tested by Kolmogorov-Smirnov Z test yielding p-values of 0.777 and 0.895 for euploid and trisomy 21 data, respectively (a normal distribution). Preferably, a genomic DNA sequence useful for screening for aneuploidies yields, using this alternative calculation, a MoM of at least about 1.5, preferably at least about 1.8, more preferably at least about 2.5 and most preferably at least about 3.0.

An additional preferred statistical verification analysis is to compare MoM DNA levels (e.g. for Beta-globin locus measurements) versus first trimester risk for aneuploidy, as estimated by other serum marker(s) and/or neural tube measurements. For the exemplary Beta-globin locus data from Table 1, a significant correlation between elevated MoM and first trimester risk was found (p=0.026). Preferably, a genomic DNA sequence useful for screening for aneuploidies yields a p value when compared to first trimester risk of p≦0.05, more preferably p≦0.03.

Total maternal blood DNA loci identified by the above methods are not used as a diagnostic test but rather as a screening test. Screening differs from diagnostic testing in that a positive total maternal blood DNA result does not mean that the patient has an affected fetus, but rather that the patient is in a category of sufficient risk to warrant further studies such as ultrasound or amniocentesis. Total maternal blood DNA testing using genomic sequences identified by the above methods may be carried out as a stand alone test. As a stand alone test, the genomic sequences are measured in samples representing a maternal bodily fluid and the results compared to the appropriate control euploid data sets as done in Table 1. Absolute or MoM based estimations of genomic DNA concentrations may be compared to empirically determined cut-off values to achieve a particular degree of aneuploidy detection sensitivity and false positive rate, depending on the specific genomic sequence used.

For example, a weighted log-linear regression MoM value may be calculated for a genomic locus for bodily fluid samples known to represent euploid and aneuploid fetal pregnancies (e.g. Table I and FIG. 3). Retrospective analysis may be used to determine the sensitivity (i.e. the MoM value that detects a certain percentage of aneuploid samples) and rate of false positives (i.e. the MoM value that detects a certain percentage of aneuploid samples but also falsely categorizes a certain percentage of euploid samples as aneuploid). The balance between sensitivity and false positives is generally determined based on clinical criteria, but 5% or 3% false positive rates are commonly accepted for other non-invasive aneuploidy screening criteria. For the example Beta-globin locus described above, a weighted log-linear regression MoM value of 3.0 correlates with a detection rate of 35% with a 5% false positive rate. Alternatively, total maternal blood DNA screening may be combined in a multivariate analysis to enhance the sensitivity and/or decrease the rate of false positives.

Improved Indirect Risk Assessments for Aneuploidy

Genomic sequences statistically verified as producing useful degrees of differentiation between samples representing euploid and aneuploid fetus pregnancies may be used alone or in conjunction with other measurements. One method of integrating the genomic DNA data produced by the methods herein is described in detail in Farina, A. et al., “Evaluation of Cell-free Fetal DNA as a Second-Trimester Maternal Serum Marker of Down Syndrome Pregnancy.” Clinical Chemistry 49:2 239-242 (2003) and the statistical techniques for integrating such data into pre-existing multivariate analysis are hereby specifically incorporated by reference.

Other methods for integrating risk evaluations derived using the genomic DNA quatitation methods disclosed herein are described generally in Section 2: Statistical Aspects of Screening for Down Syndrome, “Screening for Down Syndrome in the First Trimester,” ed. J. G. Grudzinskas and R. H. T. Ward, RCOG Press (1997), ISBN 0-902331-98-1, hereby specifically incorporated by reference. In particular, the diagnostic genomic sequences identified by the methods herein and the data produced using those sequences, as described herein, may be integrated with other predictive markers using art recognized methodologies including the Standard Likelihood Ratio Method (SLRM), the Glasgow Ratio Method (GRM), and/or other art recognized statistical methods.

All references within this application are hereby incorporated by reference in their entireties including:

Anker, P., Mulcahy, H., Chen, X. Q., and Stroun, M. (1999). Detection of circulating tumour DNA in the blood (plasma/serum) of cancer patients. Cancer Metastasis Rev. 18, 65-73.

Bauer, M., Hutterer, G., Eder, M., Majer, S., Leshane, E., Johnson, K. L., Peter, I., Bianchi, D. W., and Pertl, B. (2006). A prospective analysis of cell-free fetal DNA concentration in maternal plasma as an indicator for adverse pregnancy outcome. Prenat. Diagn. 26, 831-836.

Bianchi, D. W., Simpson, J. L., Jackson, L. G., Elias, S., Holzgreve, W., Evans, M. I., Dukes, K. A., Sullivan, L. M., Klinger, K. W., Bischoff, F. Z., Hahn, S., Johnson, K. L., Lewis, D., Wapner, R. J., and de la, C. F. (2002). Fetal gender and aneuploidy detection using fetal cells in maternal blood: analysis of NIFTY I data. National Institute of Child Health and Development Fetal Cell Isolation Study. Prenat. Diagn. 22, 609-615.

Bianchi, D. W., Williams, J. M., Sullivan, L. M., Hanson, F. W., Klinger, K. W., and Shuber, A. P. (1997). PCR quantitation of fetal cells in maternal blood in normal and aneuploid pregnancies. Am. J. Hum. Genet. 61, 822-829.

Bischoff, F. Z., Dang, D. X., Marquez-Do, D., Martinez, D., Horne, C., Lewis, D. E., and Simpson, J. L. (2003). Detecting fetal DNA from dried maternal blood spots: another step towards broad scale non-invasive prenatal genetic screening and feasible testing. Reprod. Biomed. Online. 6, 349-351.

Bischoff, F. Z., Lewis, D. E., and Simpson, J. L. (2005). Cell-free fetal DNA in maternal blood: kinetics, source and structure. Hum. Reprod. Update 11, 59-67.

Bischoff, F. Z., Nguyen, D. D., Marquez-Do, D., Moise, K. J., Jr., Simpson, J. L., and Elias, S. (1999). Noninvasive determination of fetal RhD status using fetal DNA in maternal serum and PCR. J. Soc. Gynecol. Investig. 6, 64-69.

Bischoff, F. Z., Sinacori, M. K., Dang, D. D., Marquez-Do, D., Horne, C., Lewis, D. E., and Simpson, J. L. (2002). Cell-free fetal DNA and intact fetal cells in maternal blood circulation: implications for first and second trimester non-invasive prenatal diagnosis. Hum. Reprod. Update 8, 493-500.

Chuang, D. M., Hough, C., and Senatorov, V. V. (2005). Glyceraldehyde-3-phosphate dehydrogenase, apoptosis, and neurodegenerative diseases. Annu. Rev. Pharmacol. Toxicol. 45, 269-290.

Cumming, R. C. and Schubert, D. (2005). Amyloid-Beta induces disulfide bonding and aggregation of GAPDH in Alzheimer's disease. FASEB J. 19, 2060-2062.

de la Monte, S. M. and Bloch, K. D. (1997). Aberrant expression of the constitutive endothelial nitric oxide synthase gene in Alzheimer disease. Mol. Chem. Neuropathol. 30, 139-159.

de la Monte, S. M., Sohn, Y. K., Ganju, N., and Wands, J. R. (1998). P53- and CD95-associated apoptosis in neurodegenerative diseases. Lab Invest 78, 401-411.

de la Monte, S. M., Xu, Y. Y., Hutchins, G. M., and Wands, J. R. (1996). Developmental patterns of neuronal thread protein gene expression in Down syndrome. J. Neurol. Sci. 135, 118-125.

de la, C. F., Shifrin, H., Elias, S., Bianchi, D. W., Jackson, L., Evans, M. I., Simpson, J. L., Holzgreve, W., and Klinger, K. (1998). Low false-positive rate of aneuploidy detection using fetal cells isolated from maternal blood. Fetal Diagn. Ther. 13, 380.

de la, C. F., Shifrin, H., Elias, S., Simpson, J. L., Jackson, L., Klinger, K., Bianchi, D. W., Kaplan, S. H., Evans, M. I., Holzgreve, W., and (1995). Prenatal diagnosis by use of fetal cells isolated from maternal blood. Am. J. Obstet. Gynecol. 173, 1354-1355.

Elias, S., Price, J., Dockter, M., Wachtel, S., Tharapel, A., Simpson, J. L., and Klinger, K. W. (1992). First trimester prenatal diagnosis of trisomy 21 in fetal cells from maternal blood. Lancet 340, 1033.

Farina, A., LeShane, E. S., Lambert-Messerlian, G. M., Canick, J. A., Lee, T., Neveux, L. M., Palomaki, G. E., and Bianchi, D. W. (2003). Evaluation of cell-free fetal DNA as a second-trimester maternal serum marker of Down syndrome pregnancy. Clin. Chem. 49, 239-242.

Farina, A., Sekizawa, A., Iwasaki, M., Matsuoka, R., Ichizuka, K., and Okai, T. (2004). Total cell-free DNA (Beta-globin gene) distribution in maternal plasma at the second trimester: a new prospective for preeclampsia screening. Prenat. Diagn. 24, 722-726.

Groner, Y., Elroy-Stein, O., Avraham, K. B., Schickler, M., Knobler, H., Minc-Golomb, D., Bar-Peled, O., Yarom, R., and Rotshenker, S. (1994). Cell damage by excess CuZnSOD and Down's syndrome. Biomed. Pharmacother. 48, 231-240.

Groner, Y., Elroy-Stein, O., Avraham, K. B., Yarom, R., Schickler, M., Knobler, H., and Rotman, G. (1990). Down syndrome clinical symptoms are manifested in transfected cells and transgenic mice overexpressing the human Cu/Zn-superoxide dismutase gene. J. Physiol (Paris) 84, 53-77.

Hromadnikova, I., Houbova, B., Hridelova, D., Voslarova, S., Calda, P., Nekolarova, K., Kofer, J., Stejskal, D., Doucha, J., Cinek, O., and Vavrirec, J. (2002). Quantitative analysis of DNA levels in maternal plasma in normal and Down syndrome pregnancies. BMC. Pregnancy Childbirth. 2, 4.

Johnson, K. L., Dukes, K. A., Vidaver, J., LeShane, E. S., Ramirez, I., Weber, W. D., Bischoff, F. Z., Hahn, S., Sharma, A., Dang, D. X., Hire, L. M., Bianchi, D. W., Simpson, J. L., Holzgreve, W., Elias, S., and Klinger, K. W. (2004). Interlaboratory comparison of fetal male DNA detection from common maternal plasma samples by real-time PCR. Clin. Chem. 50, 516-521.

Jorgez, C. J., Simpson, J. L., and Bischoff, F. Z. (2006). Recovery and amplification of placental RNA from dried maternal blood spots: utility for non-invasive prenatal diagnosis. Reprod. Biomed. Online. 13, 558-561.

Kamat, A. A., Bischoff, F. Z., Dang, D., Baldwin, M. F., Han, L. Y., Lin, Y. G., Merritt, W. M., Landen, C. N., Lu, C., Gershenson, D. M., Simpson, J. L., and Sood, A. K. (2006). Circulating Cell-Free DNA: A Novel Biomarker for Response to Therapy in Ovarian Carcinoma. Cancer Biol. Ther. 5.

Korenberg, J. R., Chen, X. N., Schipper, R., Sun, Z., Gonsky, R., Gerwehr, S., Carpenter, N., Daumer, C., Dignan, P., Disteche, C., and. (1994). Down syndrome phenotypes: the consequences of chromosomal imbalance. Proc. Natl. Acad. Sci. U.S.A 91, 4997-5001.

Lee, T., LeShane, E. S., Messerlian, G. M., Canick, J. A., Farina, A., Heber, W. W., and Bianchi, D. W. (2002). Down syndrome and cell-free fetal DNA in archived maternal serum. Am. J. Obstet. Gynecol. 187, 1217-1221.

Lo, Y. M., Lau, T. K., Zhang, J., Leung, T. N., Chang, A. M., Hjelm, N. M., Elmes, R. S., and Bianchi,D. W. (1999). Increased fetal DNA concentrations in the plasma of pregnant women carrying fetuses with trisomy 21. Clin. Chem. 45, 1747-1751.

Lo, Y. M., Tein, M. S., Lau, T. K., Haines, C. J., Leung, T. N., Poon, P. M., Wainscoat, J. S., Johnson, P. J., Chang, A. M., and Hjelm, N. M. (1998). Quantitative analysis of fetal DNA in maternal plasma and serum: implications for noninvasive prenatal diagnosis. Am. J. Hum. Genet. 62, 768-775.

Malone, F. D., Canick, J. A., Ball, R. H., Nyberg, D. A., Comstock, C. H., Bukowski, R., Berkowitz, R. L., Gross, S. J., Dugoff L., Craigo, S. D., Timor-Tritsch, I. E., Carr, S. R., Wolfe, H. M., Dukes, K., Bianchi, D. W., Rudnicka, A. R., Hackshaw, A. K., Lambert-Messerlian, G., Wald, N. J., and D'Alton, M. E. (2005). First-trimester or second-trimester screening, or both, for Down's syndrome. N. Engl. J. Med. 353, 2001-2011.

Marks, A., O'Hanlon, D., Lei, M., Percy, M. E., and Becker, L. E. (1996). Accumulation of S100 Beta mRNA and protein in cerebellum during infancy in Down syndrome and control subjects. Brain Res. Mol. Brain Res. 36, 343-348.

Mei, J. V., Alexander, J. R., Adam, B. W., and Hannon, W. H. (2001). Use of filter paper for the collection and analysis of human whole blood specimens. J. Nutr. 131, 1631S-1636S.

Ohashi, Y., Miharu, N., Honda, H., Samura, O., and Ohama, K. (2001). Quantitation of fetal DNA in maternal serum in normal and aneuploid prenancies. Hum. Genet. 108, 123-127.

Oyama, F., Cairns, N. J., Shimada, H., Oyama, R., Titani, K., and Ihara, Y. (1994). Down's syndrome: up-regulation of Beta-amyloid protein precursor and tau mRNAs and their defective coordination. J. Neurochem. 62, 1062-1066.

Price, J. O., Elias, S., Wachtel, S. S., Klinger, K., Dockter, M., Tharapel, A., Shulman, L. P., Phillips, O. P., Meyers, C. M., Shook, D., and (1991). Prenatal diagnosis with fetal cells isolated from maternal blood by multiparameter flow cytometry. Am. J. Obstet. Gynecol. 165, 1731-1737.

Sawa, A. (2001). Alteration of gene expression in Down's syndrome (DS) brains: its significance in neurodegeneration. J. Neural Transm. Suppl 361-371.

Seidl, R., Fang-Kircher, S., Bidmon, B., Cairns, N., and Lubec, G. (1999). Apoptosis-associated proteins p53 and APO-1/Fas (CD95) in brains of adult patients with Down syndrome. Neurosci. Lett. 260, 9-12.

Sekizawa, A., Farina, A., Koide, K., Iwasaki, M., Honma, S., Ichizuka, K., Saito, H., and Okai, T. (2004). Beta-globin DNA in maternal plasma as a molecular marker of pre-eclampsia. Prenat. Diagn. 24, 697-700.

Simpson, J. L. (2005). Choosing the best prenatal screening protocol. N. Engl. J. Med. 353, 2068-2070.

Spencer, K., De Kok, J. B., and Swinkels, D. W. (2003). Increased total cell-free DNA in the serum of pregnant women carrying a fetus affected by trisomy 21. Prenat. Diagn. 23, 580-583.

Sumarsono, S. H., Wilson, T. J., Tymms, M. J., Venter, D. J., Corrick, C. M., Kola, R., Lahoud, M. H., Papas, T. S., Seth, A., and Kola, I. (1996). Down's syndrome-like skeletal abnormalities in Ets2 transgenic mice. Nature 379, 534-537.

Swinkels D W, de Kok J B, Hendriks J C, Wiegerinck E, Zusterzeel P L, Steegers E A. (2002). Hemolysis, elevated liver enzymes, and low platelet count (HELLP) syndrome as a complication of preeclampsia in pregnant women increases the amount of cell-free fetal and maternal DNA in maternal plasma and serum. Clin Chem. 2002;48(4):650-3.

Tong, Y. K. and Lo, Y. M. (2006). Diagnostic developments involving cell-free (circulating) nucleic acids. Clin. Chim. Acta 363, 187-196.

Weitzdoerfer, R., Fountoulakis, M., and Lubec, G. (2002). Reduction of actin-related protein complex ⅔ in fetal Down syndrome brain. Biochem. Biophys. Res. Commun. 293, 836-841.

Wolvetang, E. J., Wilson, T. J., Sanij, E., Busciglio, J., Hatzistavrou, T., Seth, A., Hertzog, P. J., and Kola, I. (2003). ETS2 overexpression in transgenic models and in Down syndrome predisposes to apoptosis via the p53 pathway. Hum. Mol. Genet. 12, 247-255.

Zhong, X. Y., Burk, M. R., Troeger, C., Jackson, L. R., Holzgreve, W., and Hahn, S. (2000). Fetal DNA in maternal plasma is elevated in pregnancies with aneuploid fetuses. Prenat. Diagn. 20, 795-798.

Current Protocols in Molecular Biology, Supplement 73 (online February 2006) Ausubel et al.(ed.) John Wiley & Sons. Inc.

What is claimed is:

1. A method of identifying genomic DNA sequences that are useful for estimating the probability of an aneuploid pregnancy, the method comprising the steps of
a) estimating a total concentration of a non-Y chromosome DNA sequence in a bodily fluid sample representing an aneuploid fetal pregnancy,
b) estimating a total concentration of a non-Y chromosome DNA sequence in a bodily fluid sample representing a euploid fetal pregnancy, and
c) comparing the total concentration estimates for the DNA sequence(s) to determine whether the estimated concentration representing a euploid fetal pregnancy are significantly different from the estimated concentration representing an aneuploid fetal pregnancy.

2. The method of claim 1, wherein the bodily fluid is whole blood.

3. The method of claim 2 further comprising the step of purifying the genomic DNA from the sample.

4. The method of claim 3, wherein the total concentration of a non-Y chromosome DNA sequence is estimated using real time polymerase chain reaction.

5. The method of claims 1 wherein the comparison in 1c) comprises one or more step(s) of
a) comparing the mean values and their standard deviations, or
b) comparing the median values of the total concentration estimates for the DNA sequence(s) to derive a Multiplicity of the Median value for the total concentration of a non-Y chromosome DNA sequence in a bodily fluid sample representing an aneuploid fetal pregnancy.

6. The method of claim 5, wherein, in 5b), the total concentration estimates are first log converted and the conversion to a Multiplicity of the Median value is performed by using a weighted log-linear regression.

7. The method of claim 1, further comprising the steps of
a) comparing the total concentration estimates for the DNA sequence(s) to a predetermined first trimester risk estimate for aneuploidy, and
b) determining if the total DNA concentration estimates are significantly correlated with the predetermined first trimester risk estimate.

8. A method of estimating the probability of a test bodily fluid sample representing an aneuploid fetal pregnancy, the method comprising the steps of
a) estimating a total concentration of a non-Y chromosome DNA sequence in the test bodily fluid sample,
b) comparing the estimated concentration with a control data set representing an expected total concentration of a non-Y chromosome DNA sequence in bodily fluid samples representing euploid fetal pregnancy, and
c) determining, based on the comparison in step b), a probability that the test bodily fluid sample represents an aneuploid fetal pregnancy.

9. The method of claim 8, wherein the control data set is matched to the test bodily fluid sample for one or more of gestational age, maternal age, maternal weight, maternal diabetic status, maternal smoking status, prior maternal history of aneuploid pregnancy and maternal race.

10. The method of claims 8 wherein
a) the comparison in 8b) is performed by calculating the Multiplicity of the Median for the total concentration of a non-Y chromosome DNA sequence in the test bodily fluid sample, and
b) the determination in 8c) is based on whether the Multiplicity of the Median value meets or exceeds a threshold value which has been empirically determined to correspond to a probability of aneuploidy.

11. The method of claim 10 wherein the Multiplicity of the Median value corresponding to a probability of aneuploidy is
a) at least about 1.3 if calculated directly, or
b) at least about 1.5 if calculated using log converted data and a weighted log-linear regression.

12. The methods of claim 8, wherein the non-Y chromosome DNA sequence in the test bodily fluid sample comprises a Beta-globin genomic locus sequence.

13. The method of claim 12, wherein the Beta-globin genomic locus sequence is amplified by polymerase chain reaction primers comprising SEQ ID NO: 7 and SEQ ID NO: 8.

14. The method of claim 8, wherein the test bodily fluid sample corresponds to a first trimester pregnancy.

15. A method of estimating the probability of a bodily fluid sample representing an aneuploid fetal pregnancy, the method comprising the steps of
a) estimating a total concentration of a non-Y chromosome DNA sequence in the bodily fluid sample,
b) comparing the estimated concentration with a control data set representing an expected total concentration of a non-Y chromosome DNA sequence in bodily fluid samples representing euploid fetal pregnancy,
c) determining based on the comparison a probability that the bodily fluid sample represents an aneuploid fetal pregnancy, and
d) combining the probability determined in step c) with additional probability estimates of aneuploid fetal pregnancy to derive a combined probability estimate.


SEQUENCE LISTING (9 sequences)

SEQ ID NO: 1 - DNA, 21 bp, Artificial
  PCR PRIMER
ccccacacacatgcacttacc

SEQ ID NO: 2 - DNA, 21 bp, Artificial
  PCR PRIMER
cctagtcccagggctttgatt

SEQ ID NO: 3 - DNA, 29 bp, Artificial
  REAL TIME PCR TAQMAN ASSAY PROBE
  misc_feature (1)..(1): 6FAM
  misc_feature (31)..(31): TAMRA
aaagagctaggaaggacaggcaacttggc

SEQ ID NO: 4 - DNA, 19 bp, Artificial
  PCR PRIMER
ggtcggcgagaacctgact

SEQ ID NO: 5 - DNA, 18 bp, Artificial
  PCR PRIMER
ctgccggaggaagcaaag

SEQ ID NO: 6 - DNA, 22 bp, Artificial
  REAL TIME PCR TAQMAN ASSAY PROBE
tgcaccctcctccccaactcca

SEQ ID NO: 7 - DNA, 22 bp, Artificial
  PCR PRIMER
gtgcacctgactcctgaggaga

SEQ ID NO: 8 - DNA, 21 bp, Artificial
  PCR PRIMER
ccttgataccaacctgcccag

SEQ ID NO: 9 - DNA, 26 bp, Artificial
  REAL TIME PCR TAQMAN ASSAY PROBE
aaggtgaacgtggatgaagttggtgg